(19) United States
(12) Patent Application Publication
(10) Pub. No.: US 2026/0132146 A1
(43) Pub. Date: May 14, 2026

(54) METHODS FOR THE SYNTHESIS AND PURIFICATION OF PYROMELLITIC DIANHYDRIDE AND RELATED COMPOSITIONS

(71) Applicant: ENTEGRIS, INC., Billerica, MA (US)

(72) Inventor: David M. Ermert, Danbury, CT (US)

(21) Appl. No.: 19/383,404

(22) Filed: Nov. 7, 2025

Related U.S. Application Data
(60) Provisional application No. 63/719,535, filed on Nov. 12, 2024.

(51) Int. Cl. C07D 493/04

(52) U.S. Cl. CPC C07D 493/04

(57) ABSTRACT

Methods and related compositions for the synthesis and purification of pyromellitic dianhydride are provided. The method comprises obtaining a solid composition. The solid composition comprises at least one of a pyromellitic anhydride, a pyromellitic acid, or any combination thereof. The method comprises heating the solid composition at a temperature and a pressure sufficient to convert at least one of the pyromellitic anhydride, the pyromellitic acid, or a combination thereof to a reaction product. The reaction product comprises a pyromellitic dianhydride having a purity of at least 99.9% as measured by ¹H NMR.

### CROSS-REFERENCE TO RELATED APPLICATIONS

This application claims the benefit under 35 USC 119 of U.S. Provisional Patent Application No. 63/719,535, filed Nov. 12, 2024, the disclosure of which is hereby incorporated herein by reference in its entirety.

### FIELD

The present disclosure relates to methods for the synthesis and purification of pyromellitic dianhydride and related compositions.

### BACKGROUND

Conventional methods for synthesizing certain compounds can involve the use of reagents that negatively impact the environment. In addition, conventional methods for synthesizing certain compounds can involve various methods that do not purify the compound.

### SUMMARY

Some embodiments relate to a method. In some embodiments, the method comprises obtaining a solid composition. In some embodiments, the solid composition comprises at least one of a pyromellitic anhydride, a pyromellitic acid, or any combination thereof. In some embodiments, the method comprises heating the solid composition at a temperature and a pressure sufficient to convert at least one of the pyromellitic anhydride, the pyromellitic acid, or a combination thereof to a reaction product. In some embodiments, the reaction product comprises a pyromellitic dianhydride having a purity of at least 99.9% as measured by ¹H Proton Nuclear Magnetic Resonance (NMR) Spectroscopy.

Some embodiments relate to a method. In some embodiments, the method comprises obtaining a solid pyromellitic acid. In some embodiments, the method comprises heating the solid pyromellitic acid at a temperature and pressure sufficient to convert the pyromellitic acid to a pyromellitic dianhydride.

Some embodiments relate to a composition. In some embodiments, the composition comprises a pyromellitic dianhydride having a purity of at least 99.9% as measured by ¹H NMR.

Some embodiments relate to a method. In some embodiments, the method comprises obtaining a solid reaction product. In some embodiments, the reaction product comprises a pyromellitic dianhydride having a purity of at least 99.9% as measured by ¹H NMR. In some embodiments, the method comprises vaporizing the solid reaction product to obtain a reaction product vapor. In some embodiments, the method comprises selectively depositing the reaction product vapor on a substrate.

### BRIEF DESCRIPTION OF THE DRAWINGS

FIG. 1 is a flowchart of a method for purifying a solid composition, according to some embodiments.

FIG. 2 is a flowchart of a method for synthesizing a pyromellitic dianhydride, according to some embodiments.

FIG. 3 is a flowchart of a method for depositing a purified reaction product on a substrate, according to some embodiments.

### DETAILED DESCRIPTION

Some embodiments relate to methods for purifying and synthesizing pyromellitic dianhydride. For example, in some embodiments, the methods are useful for converting at least a portion of the impurities to a reaction product such as, for example and without limitation, a pyromellitic dianhydride, to further increase a purity of the pyromellitic dianhydride. At least some of these embodiments relate to methods for purifying and synthesizing precursors useful in the fabrication of microelectronic devices, including semiconductor devices, and the like. For example, the precursor may be used to deposit on a substrate by one or more deposition processes. Examples of deposition processes include, without limitation, at least one of a chemical vapor deposition (CVD) process, a digital or pulsed chemical vapor deposition process, a plasma-enhanced cyclical chemical vapor deposition process (PECCVD), a flowable chemical vapor deposition process (FCVD), an atomic layer deposition (ALD) process, a thermal atomic layer deposition, a plasma-enhanced atomic layer deposition (PEALD) process, a metal organic chemical vapor deposition (MOCVD) process, a plasma-enhanced chemical vapor deposition (PECVD) process, or any combination thereof.

Some embodiments relate to a composition. In some embodiments, the composition comprises a pyromellitic dianhydride having a purity of at least 99.9% as measured by proton ¹H nuclear magnetic resonance (¹H NMR).

In some embodiments, the pyromellitic dianhydride is a compound of the formula:

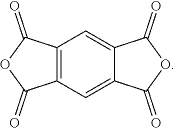 [SMILES: O=c1oc(=O)c2cc3c(=O)oc(=O)c3cc12]

In some embodiments, the pyromellitic dianhydride has a purity of at least 99.9% as measured by ¹H NMR. For example, in some embodiments, the pyromellitic dianhydride has a purity of at least 99.91%, at least 99.92%, at least 99.93%, at least 99.94%, at least 99.95%, at least 99.96%, at least 99.97%, at least 99.98%, or at least 99.99% as measured by ¹H NMR. In some embodiments, the pyromellitic dianhydride has a purity of 99.9% to 99.9999%, or any range or subrange between 99.9% to 99.9999%, as measured by ¹H NMR. In some embodiments, the pyromellitic dianhydride has a purity of 99.91% to 99.9999%, 99.92% to 99.9999%, 99.93% to 99.9999%, 99.94% to 99.9999%, 99.95% to 99.9999%, 99.96% to 99.9999%, 99.97% to 99.9999%, 99.98% to 99.9999%, 99.99% to 99.9999%, 99.991% to 99.9999%, 99.992% to 99.9999%, 99.993% to 99.9999%, 99.994% to 99.9999%, 99.995% to 99.9999%, 99.996% to 99.9999%, 99.997% to 99.9999%, 99.998% to 99.9999%, 99.999% to 99.9999%, 99.9995% to 99.9999%, 99.9% to 99.9995%, 99.9% to 99.999%, 99.9% to 99.998%, 99.9% to 99.997%, 99.9% to 99.996%, 99.9% to 99.995%, 99.9% to 99.994%, 99.9% to 99.993%, 99.9% to 99.992%, 99.9% to 99.991%, 99.9% to 99.99%, 99.9% to 99.98%, 99.9% to 99.97%, 99.9% to 99.96%, 99.9% to 99.95%, 99.9% to 99.94%, 99.9% to 99.93%, 99.9% to 99.92%, or 99.9% to 99.91%.

In some embodiments, the composition does not comprise a water, an organic acid, or an organic-based solvent. In some embodiments, the composition is free of moisture. In some embodiments, the composition is free of water. In some embodiments, the composition is free of organic acids. In some embodiments, the composition is free of organic-based solvents.

In some embodiments, the organic acids comprise at least one of an acetic acid, a formic acid, a citric acid, an oxalic acid, a uric acid, a malic acid, a tartaric acid, a butyric acid, a folic acid, a lactic acid, or any combination thereof. In some embodiments, the organic-based solvents comprise at least one species selected from the group consisting of a glycol ether (e.g., diethylene glycol monomethyl ether, a diethylene glycol monoethyl ether, diethylene glycol monobutyl ether), DMSO, a sulfolane, a methanesulfonic acid, or combinations thereof.

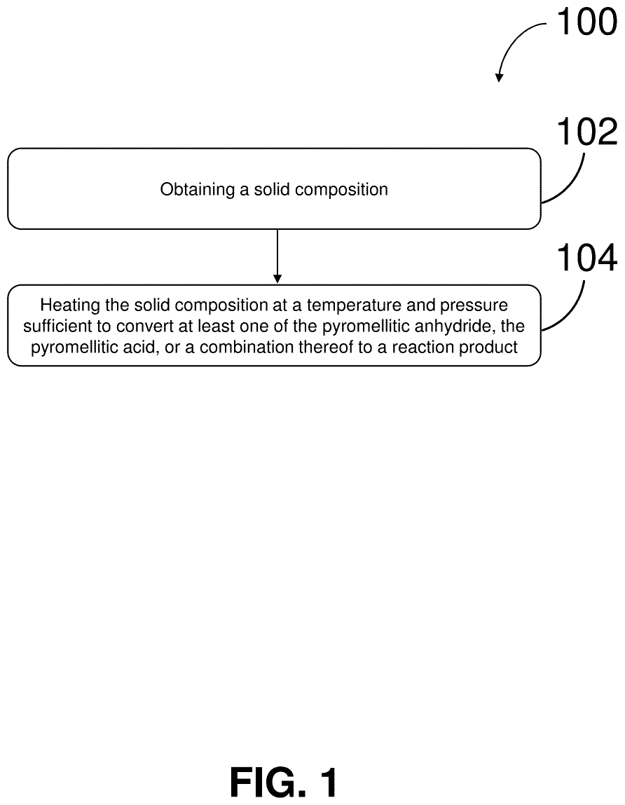

FIG. 1 is a flowchart of a method for purifying a solid composition, according to some embodiments. As shown in FIG. 1, the method for purifying a solid composition may comprise one or more of the following steps: obtaining a solid composition 102, and heating the solid composition at a temperature and a pressure sufficient to convert at least one of a pyromellitic anhydride, a pyromellitic acid, or a combination thereof to a reaction product 104. In some embodiments, the method does not comprise sublimating the solid composition 102.

At step 102, in some embodiments, the method comprises obtaining a solid composition. In some embodiments, the solid composition comprises at least one of a pyromellitic anhydride, a pyromellitic acid, or any combination thereof.

In some embodiments, the solid composition comprises a pyromellitic anhydride. In some embodiments, the pyromellitic anhydride is a compound of the formula:

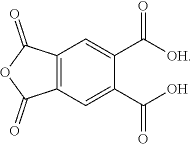 [SMILES: O=C(O)c1cc2c(cc1C(=O)O)C(=O)OC2=O]

In some embodiments, the composition comprises 0.01% to 1% by weight of the pyromellitic anhydride based on a total weight of the composition, or any range or subrange between 0.01% to 1%. For example, in some embodiments, the composition comprises 0.1% to 0.9%, 0.2% to 0.8%, 0.3% to 0.7%, or 0.4% to 0.6% by weight of the pyromellitic anhydride based on a total weight of the composition. In some embodiments, the composition comprises 0.1% to 1%, 0.2% to 1%, 0.3% to 1%, 0.4% to 1%, 0.5% to 1%, 0.6% to 1%, 0.7% to 1%, 0.8% to 1%, or 0.9% to 1% by weight of the pyromellitic anhydride based on a total weight of the composition.

In some embodiments, the solid composition comprises a pyromellitic acid. In some embodiments, the pyromellitic acid is a compound of the formula:

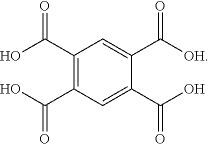 [SMILES: O=C(O)c1cc(C(=O)O)c(C(=O)O)cc1C(=O)O]

In some embodiments, the composition comprises 0.01% to 1% by weight of the pyromellitic acid based on a total weight of the composition, or any range or subrange between 0.01% to 1%. For example, in some embodiments, the composition comprises 0.1% to 0.9%, 0.2% to 0.8%, 0.3% to 0.7%, or 0.4% to 0.6% by weight of the pyromellitic acid based on a total weight of the composition. In some embodiments, the composition comprises 0.1% to 1%, 0.2% to 1%, 0.3% to 1%, 0.4% to 1%, 0.5% to 1%, 0.6% to 1%, 0.7% to 1%, 0.8% to 1%, or 0.9% to 1% by weight of the pyromellitic acid based on a total weight of the composition.

In some embodiments, the solid composition comprises a second pyromellitic dianhydride. In some embodiments, the solid composition comprises 90% to 99% of the second pyromellitic dianhydride based on a total weight of the solid composition, or any range or subrange between 90% and 99%. For example, in some embodiments, the solid composition comprises 91% to 98%, 92% to 97%, 93% to 96%, or 94% to 95% of the second pyromellitic dianhydride based on a total weight of the solid composition. In some embodiments, the solid composition comprises 91% to 99%, 92% to 99%, 93% to 99%, 94% to 99%, 95% to 99%, 96% to 99%, 97% to 99%, or 98% to 99% of the second pyromellitic dianhydride based on a total weight of the solid composition. In some embodiments, the solid composition comprises 90% to 98%, 90% to 97%, 90% to 96%, 90% to 95%, 91% to 95%, 92% to 95%, 93% to 95%, or 94% to 95% of the second pyromellitic dianhydride based on a total weight of the solid composition.

In some embodiments, the solid composition further comprises an impurity. In some embodiments, the impurity is a compound other than a pyromellitic dianhydride. In some embodiments, the impurity may be an unknown impurity. In some embodiments, the composition comprises 0.1 parts per million (ppm) to 5 ppm of the impurity based on a total weight of the composition, or any range or subrange between 0.1 ppm to 5 ppm. For example, in some embodiments, the composition comprises 0.5 ppm to 4.5 ppm, 1 ppm to 4 ppm, 1.5 ppm to 3.5 ppm, or 2 ppm to 2.5 ppm of the impurity based on the total weight of the composition. In some embodiments, the composition comprises 1 ppm to 5 ppm, 2 ppm to 5 ppm, 3 ppm to 5 ppm, or 4 ppm to 2.5 ppm of the impurity based on the total weight of the composition. In some embodiments, the composition comprises 0.1 ppm to 4 ppm, 0.1 ppm to 3 ppm, 0.1 ppm to 2 ppm, or 0.1 ppm to 1 ppm of the impurity based on the total weight of the composition.

At step 104, in some embodiments, the method comprises heating the solid composition at a temperature and a pressure sufficient to convert at least one of the pyromellitic anhydride, the pyromellitic acid, or a combination thereof to a reaction product. In some embodiments, the heating is conducted at a temperature and a pressure at which the solid composition does not substantially sublimate.

In some embodiments, the heating is conducted at a temperature of 200° C. to 300° C., or any range or subrange between 200° C. to 300° C. For example, in some embodiments, the heating is conducted at a temperature of 210° C. to 290° C., 220° C. to 280° C., 230° C. to 270° C., or 240° C. to 260° C. In some embodiments, the heating is conducted at a temperature of 210° C. to 300° C., 220° C. to 300° C., 230° C. to 300° C., 240° C. to 300° C., 250° C. to 300° C., 260° C. to 300° C., 270° C. to 300° C., 280° C. to 300° C., or 280° C. to 300° C. In some embodiments, the heating is conducted at a temperature of 200° C. to 290° C., 200° C. to 280° C., 200° C. to 270° C., 200° C. to 260° C., 200° C. to 250° C., 200° C. to 240° C., 200° C. to 230° C., 200° C. to 220° C., or 200° C. to 210° C.

In some embodiments, the heating is conducted at atmospheric pressure.

In some embodiments, the heating is conducted under an inert gas. In some embodiments, the inert gas may be an argon, a helium, a neon, a krypton, a xenon, a radon, a nitrogen, or a carbon dioxide. In some embodiments, the heating is conducted under a nitrogen atmosphere.

In some embodiments, during the heating, the impurity is converted to the reaction product.

In some embodiments, the heating is conducted for a sufficient time to convert the pyromellitic anhydride, the pyromellitic acid, the impurity, or any combination thereof to the reaction product. In some embodiments, the heating is conducted for 30 minutes to 3 hours, or any range or subrange between 30 minutes and 3 hours. In some embodiments, the heating is conducted for 1 hour to 3 hours, 1.5 hours to 3 hours, 2 hours to 3 hours, or 2.5 hours to 3 hours. In some embodiments, the heating is conducted for 30 minutes to 2.5 hours, 30 minutes to 2 hours, 30 minutes to 1.5 hours, or 30 minutes to 1 hour.

In some embodiments, during the heating, the impurity does not substantially sublimate. In some embodiments, during the heating, less than 5% of the impurity sublimates. For example, in some embodiments, during the heating, less than 5%, less than 4%, less than 3%, less than 2%, less than 1%, less than 0.5%, less than 0.1%, or less than 0.01% of the impurity sublimates.

In some embodiments, during the heating, the impurity does not sublimate.

In some embodiments, during the heating, the solid composition does not substantially sublimate. In some embodiments, during the heating, less than 5% of the solid composition sublimates. For example, in some embodiments, during the heating, less than 5%, less than 4%, less than 3%, less than 2%, less than 1%, less than 0.5%, less than 0.1%, or less than 0.01% of the solid composition sublimates.

In some embodiments, during the heating, the solid composition does not sublimate.

In some embodiments, the reaction product comprises a pyromellitic dianhydride having a purity of at least 99.9% as measured by ¹H NMR. For example, in some embodiments, the pyromellitic dianhydride has a purity of at least 99.91%, at least 99.92%, at least 99.93%, at least 99.94%, at least 99.95%, at least 99.96%, at least 99.97%, at least 99.98%, or at least 99.99% as measured by ¹H NMR.

In some embodiments, the pyromellitic dianhydride has a purity of 99.9% to 99.9999%, or any range or subrange between 99.9% to 99.9999%, as measured by ¹H NMR. In some embodiments, the pyromellitic dianhydride has a purity of 99.91% to 99.9999%, 99.92% to 99.9999%, 99.93% to 99.9999%, 99.94% to 99.9999%, 99.95% to 99.9999%, 99.96% to 99.9999%, 99.97% to 99.9999%, 99.98% to 99.9999%, 99.99% to 99.9999%, 99.991% to 99.9999%, 99.992% to 99.9999%, 99.993% to 99.9999%, 99.994% to 99.9999%, 99.995% to 99.9999%, 99.996% to 99.9999%, 99.997% to 99.9999%, 99.998% to 99.9999%, 99.999% to 99.9999%, 99.9995% to 99.9999%, 99.9% to 99.9995%, 99.9% to 99.999%, 99.9% to 99.998%, 99.9% to 99.997%, 99.9% to 99.996%, 99.9% to 99.995%, 99.9% to 99.994%, 99.9% to 99.993%, 99.9% to 99.992%, 99.9% to 99.991%, 99.9% to 99.99%, 99.9% to 99.98%, 99.9% to 99.97%, 99.9% to 99.96%, 99.9% to 99.95%, 99.9% to 99.94%, 99.9% to 99.93%, 99.9% to 99.92%, or 99.9% to 99.91%.

In some embodiments, the pyromellitic dianhydride is a compound of the formula:

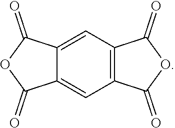 [SMILES: O=c1oc(=O)c2cc3c(=O)oc(=O)c3cc12]

In some embodiments, as measured by ¹H NMR and ¹³C NMR, the pyromellitic dianhydride, the pyromellitic anhydride, and the pyromellitic acid were determined to have chemical shifts using d₆-DMSO as shown in Table 1.

TABLE 1

| | NMR Chemical shift (δ) | |
|---|---|---|
| Compound | ¹H | ¹³C |
| Pyromellitic dianhydride | 8.75 (s, 2H) | 121.93, 137.75, 161.48 |
| Pyromellitic anhydride | 14.0 (s, 2H), 8.24 (s, 2H) | |
| Pyromellitic acid | 13.59 (s, 4H), 7.93 (s, 2H) | 128.39, 134.55, 167.25 |

In some embodiments, the method does not comprise the use of a solvent. In some embodiments, the method does not comprise the use of at least one of an organic solvent, an inorganic solvent, or any combination hereof. In some embodiments, the method does not comprise a solvent(s) selected from the group consisting of methanol, ethanol, isopropanol, butanol, pentanol, hexanol, 2-ethyl-1-hexanol, heptanol, octanol, ethylene glycol, propylene glycol, butylene glycol, butylene carbonate, ethylene carbonate, propylene carbonate, dipropylene glycol, diethylene glycol, diethylene glycol monomethyl ether, triethylene glycol monomethyl ether, diethylene glycol monoethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethylene glycol monohexyl ether, ethylene glycol phenyl ether, propylene glycol methyl ether, dipropylene glycol methyl ether (DPGME), tripropylene glycol methyl ether (TPGME), dipropylene glycol dimethyl ether, dipropylene glycol ethyl ether, propylene glycol n-propyl ether, dipropylene glycol n-propyl ether (DPGPE), tripropylene glycol n-propyl ether, propylene glycol n-butyl ether, dipropylene glycol n-butyl ether, tripropylene glycol n-butyl ether, propylene glycol phenyl ether, 2,3-dihydrodecafluoropentane, ethyl perfluorobutylether, methyl perfluorobutylether, dimethyl sulfoxide (DMSO), sulfolane, 4-methyl-2-pentanol, diethylene glycol diethyl ether, diethylene glycol ethyl methyl ether, 3-methyl-2-oxazolidone, N-methylmorpholine N-oxide, trimethylamine N-oxide and combinations thereof. It will be appreciated that other solvents may not be used herein without departing from this disclosure.

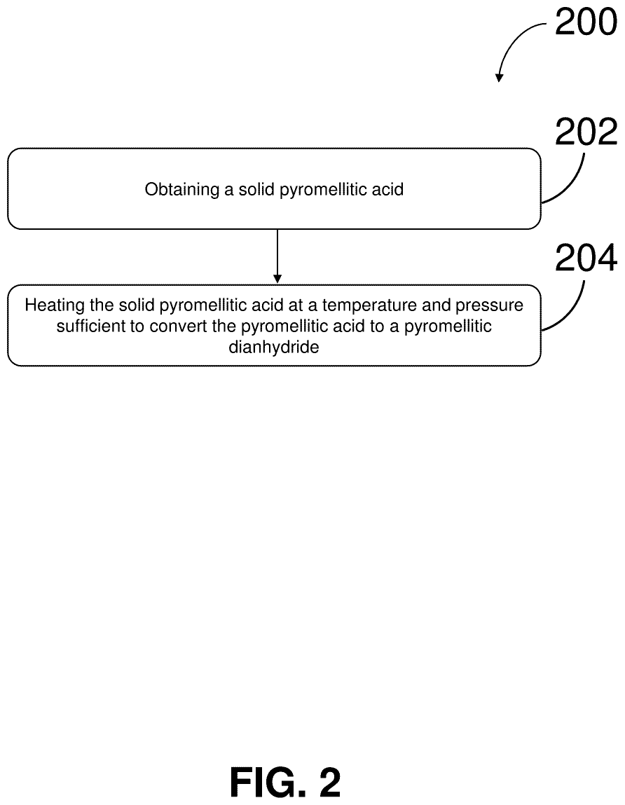

FIG. 2 is a flowchart of a method for synthesizing a pyromellitic dianhydride, according to some embodiments. As shown in FIG. 2, the method for synthesizing a pyromellitic dianhydride 200 may comprise one or more of the following steps: obtaining a solid pyromellitic acid 202, and heating the solid pyromellitic acid at a temperature and pressure sufficient to convert the pyromellitic acid to a pyromellitic dianhydride 204.

At step 202, in some embodiments, the method comprises obtaining a solid pyromellitic acid.

In some embodiments, the solid pyromellitic acid is a compound of the formula:

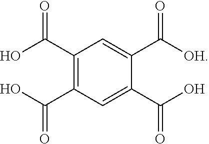 [SMILES: O=C(O)c1cc(C(=O)O)c(C(=O)O)cc1C(=O)O]

At step 204, in some embodiments, the method comprises heating the solid pyromellitic acid at a temperature and pressure sufficient to convert the pyromellitic acid to a pyromellitic dianhydride. In some embodiments, the heating is conducted at a temperature and a pressure at which the solid pyromellitic acid does not substantially sublimate.

In some embodiments, the heating is conducted at a temperature of 200° C. to 300° C., or any range or subrange between 200° C. and 300° C. For example, in some embodiments, the heating is conducted at a temperature of 210° C. to 290° C., 220° C. to 280° C., 230° C. to 270° C., or 240° C. to 260° C. In some embodiments, the heating is conducted at a temperature of 210° C. to 300° C., 220° C. to 300° C., 230° C. to 300° C., 240° C. to 300° C., 250° C. to 300° C., 260° C. to 300° C., 270° C. to 300° C., 280° C. to 300° C., or 290° C. to 300° C.

In some embodiments, the heating is conducted for 1 hour. In some embodiments, the heating is conducted for 30 minutes to 6 hours, or any range or subrange between 30 minutes and 6 hours. For example, in some embodiments, the heating is conducted for 1 hour to 5.5 hours, 1.5 hours to 5 hours, 2 hours to 4.5 hours, 2.5 hours to 4 hours, or 3 hours to 3.5 hours. In some embodiments, the heating is conducted for 30 minutes to 5.5 hours, 30 minutes to 5 hours, 30 minutes to 4.5 hours, 30 minutes to 4 hours, 30 minutes to 3.5 hours, 30 minutes to 3 hours, 30 minutes to 2.5 hours, 30 minutes to 2 hours, 30 minutes to 1.5 hours, or 30 minutes to 1 hours. In some embodiments, the heating is conducted for 1 hour to 6 hours, 1.5 hours to 6 hours, 2 hours to 6 hours, 2.5 hours to 6 hours, 3 hours to 6 hours, 3.5 hours to 6 hours, 4 hours to 6 hours, 4.5 hours to 6 hours, 5 hours to 6 hours, or 5.5 hours to 6 hours.

In some embodiments, the heating is conducted at atmospheric pressure. In some embodiments, the heating is conducted under an inert gas. In some embodiments, the heating is conducted at an elevated pressure. In some embodiments, the elevated pressure is a pressure above atmospheric pressure. In some embodiments, the heating is conducted under a nitrogen atmosphere.

In some embodiments, at least 99.9% of the solid pyromellitic acid is converted to the pyromellitic dianhydride. For example, in some embodiments, at least 99.91%, at least 99.92%, at least 99.93%, at least 99.94%, at least 99.95%, at least 99.96%, at least 99.97%, at least 99.98%, or at least 99.9% of the solid pyromellitic acid is converted to the pyromellitic dianhydride.

In some embodiments, 99.9% to 100% of the solid pyromellitic acid is converted to the pyromellitic dianhydride, or any range or subrange between 99.9% and 100%. In some embodiments, 99.91% to 99.99%, 99.92% to 99.98%, 99.93% to 99.97%, or 99.94% to 99.96% of the solid pyromellitic acid is converted to the pyromellitic dianhydride. In some embodiments, 99.9% to 99.99%, 99.9% to 99.98%, 99.9% to 99.97%, 99.9% to 99.96%, 99.9% to 99.95%, 99.9% to 99.94%, 99.9% to 99.93%, 99.9% to 99.92%, or 99.9% to 99.91% of the solid pyromellitic acid is converted to the pyromellitic dianhydride. In some embodiments, 99.91% to 100%, 99.92% to 100%, 99.93% to 100%, 99.94% to 100%, 99.95% to 100%, 99.96% to 100%, 99.97% to 100%, 99.98% to 100%, or 99.99% to 100% of the solid pyromellitic acid is converted to the pyromellitic dianhydride.

In some embodiments, during the heating, the solid pyromellitic acid does not substantially sublimate. In some embodiments, during the heating, less than 5%, less than 4%, less than 3%, less than 2%, less than 1%, less than 0.5%, less than 0.1%, or less than 0.01% of the solid pyromellitic acid sublimates.

In some embodiments, during the heating, the solid pyromellitic acid does not sublimate.

In some embodiments, the method does not comprise the use of a solvent, as described herein.

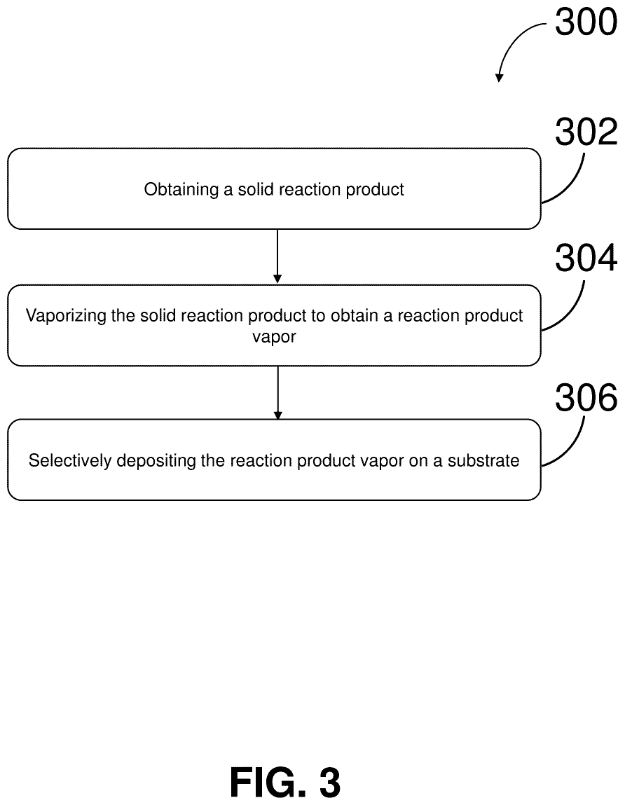

FIG. 3 is a flowchart of a method for depositing a purified reaction product on a substrate, according to some embodiments. As shown in FIG. 3, the method for depositing a purified reaction product on a substrate 300 may comprise one or more of the following steps: obtaining a solid reaction product 302, vaporizing the solid reaction product to obtain a reaction product vapor 304, and selectively depositing the reaction product vapor on a substrate 306.

At step 302, in some embodiments, the method comprises obtaining a solid reaction product. In some embodiments, the reaction product comprises a pyromellitic dianhydride having a purity of at least 99.9% as measured by ¹H NMR, as described herein.

At step 304, in some embodiments, the method comprises vaporizing the solid reaction product to obtain a reaction product vapor.

In some embodiments, the vaporizing may comprise heating the solid reaction product. In some embodiments, the vaporizing may comprise heating to a temperature below a decomposition temperature of the solid reaction product.

At step 306, in some embodiments, the method comprises selectively depositing the reaction product vapor on a substrate. In some embodiments, the selectively depositing comprises a physical vapor deposition process. In some embodiments, the selectively depositing comprises at least one of a CVD process, a digital or pulsed chemical vapor deposition process, a PECCVD process, a FCVD process, an ALD process, a thermal atomic layer deposition, PEALD process, a MOCVD process, a PECVD process, or any combination thereof. In some embodiments, the selectively depositing comprises a CVD process. In some embodiments, the selectively depositing comprises a PECCVD process. In some embodiments, the selectively depositing comprises a FCVD process. In some embodiments, the selectively depositing comprises an ALD process. In some embodiments, the selectively depositing comprises a thermal atomic layer deposition. In some embodiments, the selectively depositing comprises a PEALD process. In some embodiments, the selectively depositing comprises a MOCVD process. In some embodiments, the selectively depositing comprises a PECVD process.

In some embodiments, the substrate comprises a cobalt, a ruthenium, or any combination thereof. In some embodiments, the substrate comprises a cobalt. In some embodiments, the substrate comprises a ruthenium.

Any one or more of the embodiments disclosed herein shall be understood to be combinable without departing from the scope or spirit of the disclosure.

### Example 1

A solid composition comprising 99% by weight of pyromellitic dianhydride and 1% by weight of pyromellitic anhydride, pyromellitic acid, and other impurities based on a total weight of the composition was heated at 200° C. to 300° C. for 1 to 3 hours. The resulting solid composition was measured using ¹H-NMR and ¹³C-NMR and was found to comprise 99.96% by weight of pyromellitic dianhydride by the presence of a chemical shift at 8.75 using ¹H-NMR and the presence of chemical shifts at 121.93, 137.75, 161.48 using ¹³C-NMR. No substantial sublimation was observed.

### Example 2

A solid composition of pyromellitic acid was heated at 200° C. to 300° C. for 1 to 3 hours to form a solid composition of pyromellitic dianhydride. The reaction product was confirmed as pyromellitic dianhydride using ¹H NMR and ¹³C-NMR. 100% of the pyromellitic acid was converted to pyromellitic dianhydride as measured by the presence of a chemical shift at 8.75 using ¹H-NMR and the presence of chemical shifts at 121.93, 137.75, 161.48 using ¹³C-NMR. No substantial sublimation was observed.

### Aspects

Various Aspects are described below. It is to be understood that any one or more of the features recited in the following Aspect(s) can be combined with any one or more other Aspect(s).

Aspect 1. A method comprising:
obtaining a solid composition,
   wherein the solid composition comprises at least one of:
      a pyromellitic anhydride;
      a pyromellitic acid; or
      any combination thereof; and
heating the solid composition at a temperature and a pressure sufficient to convert at least one of the pyromellitic anhydride, the pyromellitic acid, or any combination thereof to a reaction product,
   wherein the reaction product comprises a pyromellitic dianhydride having a purity of at least 99.9% as measured by ¹H NMR.
      Aspect 2. The method according to Aspect 1, wherein the heating is conducted at a temperature of 200° C. to 300° C.
      Aspect 3. The method according to any one of Aspects 1-2, wherein the heating is conducted at atmospheric pressure.
      Aspect 4. The method according to any one of Aspects 1-3, wherein the heating is conducted under a nitrogen atmosphere.
      Aspect 5. The method according to any one of Aspects 1-4, wherein the solid composition comprises a second pyromellitic dianhydride.
      Aspect 6. The method according to any one of Aspects 1-5, wherein the solid composition further comprises an impurity.
      Aspect 7. The method according to any one of Aspects 1-6, wherein, during the heating, the impurity is converted to the reaction product.
      Aspect 8. The method according to any one of Aspects 1-7, wherein, during the heating, the impurity does not substantially sublimate.
      Aspect 9. The method according to any one of Aspects 1-8, wherein, during the heating, the solid composition does not substantially sublimate.
      Aspect 10. A method comprising:
obtaining a solid pyromellitic acid; and
heating the solid pyromellitic acid at a temperature and pressure sufficient to convert the pyromellitic acid to a pyromellitic dianhydride.
   Aspect 11. The method according to Aspect 10, wherein the heating is conducted at a temperature of 200° C. to 300° C.
   Aspect 12. The method according to any one of Aspects 10-11, wherein the heating is conducted at atmospheric pressure.
   Aspect 13. The method according to any one of Aspects 10-12, wherein the heating is conducted under a nitrogen atmosphere.
   Aspect 14. The method according to any one of Aspects 10-13, wherein the heating is conducted at an elevated pressure.
   Aspect 15. The method according to any one of Aspects 10-14, wherein at least 99.9% of the solid pyromellitic acid is converted to the pyromellitic dianhydride.
   Aspect 16. The method according to any one of Aspects 10-15, wherein the method does not comprise the use of a solvent.
   Aspect 17. The method according to any one of Aspects 10-16, wherein, during the heating, the solid pyromellitic acid does not substantially sublimate.
   Aspect 18. A solid composition comprising:
a pyromellitic dianhydride having a purity of at least 99.9% as measured by ¹H NMR.
   Aspect 19. The composition according to Aspect 18, wherein the composition does not comprise a water, an organic acid, or an organic-based solvent.
   Aspect 20. A method comprising:
obtaining a solid reaction product,
   wherein the reaction product comprises a pyromellitic dianhydride having a purity of at least 99.9% as measured by ¹H NMR;
vaporizing the solid reaction product to obtain a reaction product vapor; and selectively depositing the reaction product vapor on a substrate.
   Aspect 21. The method according to Aspect 19, wherein the substrate comprises a cobalt, a ruthenium, or any combination thereof.

What is claimed is:

1. A method comprising:
obtaining a solid composition,
wherein the solid composition comprises at least one of:
a pyromellitic anhydride;
a pyromellitic acid; or
any combination thereof; and
heating the solid composition at a temperature and a pressure sufficient to convert at least one of the pyromellitic anhydride, the pyromellitic acid, or any combination thereof to a reaction product,
wherein the reaction product comprises a pyromellitic dianhydride having a purity of at least 99.9% as measured by ¹H NMR.

2. The method of claim 1, wherein the heating is conducted at a temperature of 200° C. to 300° C.

3. The method of claim 1, wherein the heating is conducted at atmospheric pressure.

4. The method of claim 1, wherein the heating is conducted under a nitrogen atmosphere.

5. The method of claim 1, wherein the solid composition comprises a second pyromellitic dianhydride.

6. The method of claim 1, wherein the solid composition further comprises an impurity.

7. The method of claim 6, wherein, during the heating, the impurity is converted to the reaction product.

8. The method of claim 6, wherein, during the heating, the impurity does not substantially sublimate.

9. The method of claim 1, wherein, during the heating, the solid composition does not substantially sublimate.

10. A method comprising:
obtaining a solid pyromellitic acid; and
heating the solid pyromellitic acid at a temperature and pressure sufficient to convert the pyromellitic acid to a pyromellitic dianhydride.

11. The method of claim 10, wherein the heating is conducted at a temperature of 200° C. to 300° C.

12. The method of claim 10, wherein the heating is conducted at atmospheric pressure.

13. The method of claim 10, wherein the heating is conducted under a nitrogen atmosphere.

14. The method of claim 10, wherein the heating is conducted at an elevated pressure.

15. The method of claim 10, wherein at least 99.9% of the solid pyromellitic acid is converted to the pyromellitic dianhydride.

16. The method of claim 10, wherein the method does not comprise the use of a solvent.

17. The method of claim 10, wherein, during the heating, the solid pyromellitic acid does not substantially sublimate.

18. A composition comprising:
a precursor,
wherein the precursor comprises a pyromellitic dianhydride having a purity of at least 99.9% as measured by ¹H NMR.

19. The composition of claim 18, wherein the composition does not comprise a water, an organic acid, or an organic-based solvent.

20. A method comprising:
obtaining a solid reaction product,
wherein the reaction product comprises a pyromellitic dianhydride having a purity of at least 99.9% as measured by ¹H NMR;
vaporizing the solid reaction product to obtain a reaction product vapor, and
selectively depositing the reaction product vapor on a substrate.

21. The method of claim 20, wherein the substrate comprises a cobalt, a ruthenium, or any combination thereof.
